(19) United States
(12) Patent Application Publication
(10) Pub. No.: US 2007/0060829 A1
(43) Pub. Date: Mar. 15, 2007

(54) Method of finding the source of and treating cardiac arrhythmias

(76) Inventor: Carlo Pappone, Milano (IT)

Correspondence Address: Edward Renner, Suite 400, 7700 Bonhomme, St. Louis, MO, 63105, US

(21) Appl. No.: 11/478,441

(22) Filed: Jun. 29, 2006

Related U.S. Application Data
(60) Provisional application No. 60/701,226, filed on Jul. 21, 2005.

(51) Int. Cl. A61B 5/04

(52) U.S. Cl. 600/509

(57) ABSTRACT

A method of mapping ventricular arrhythmias in a subject includes placing a plurality of ECG leads on the subject; triggering an arrhythmia with a pacing catheter, determining its location and recording the resulting ECG signals from the plurality of leads; successively navigating the electrode on a catheter to each of a plurality of points in the general location of the source of the arrhythmia site, applying electrical stimulation to each site via the electrode, and recording the resulting paced ECG; and comparing each paced ECG from each point with an ECG during the arrhythmia to identify the point where the paced ECG most closely corresponds to the ECG during arrhythmia.

### CROSS-REFERENCE TO RELATED APPLICATIONS

This application claims the benefit of U.S. Provisional Patent Application Ser. No. 60/701,226, filed Jul. 21, 2005, the entire disclosure of which is incorporated herein by reference.

### BACKGROUND OF THE INVENTION

This invention relates to diagnosing and treating cardiac arrhythmias, and in particular to a method of finding the source of a cardiac arrhythmia and treating the arrhythmia.

Cardiac arrhythmias are usually the result of errant electrical signals in the heart. There are several treatments for arrhythmias, including drugs, surgical procedures in which blocks to the errant electrical signals are made by making incisions in the heart tissue, and ablation procedures which attempt to destroy the source of the errant signals or block the paths of the errant signals. One of the difficulties of these ablation procedures is accurately identifying the source of the errant signals. A physician can manipulate the mapping catheter over the cardiac surfaces to sense the local electrical signals and try to identify the source of the signal. This can be a time consuming and tedious task. Remote navigations systems have made this somewhat better, by allowing for the automated mapping of selected cardiac surfaces. The remote navigation systems are usually mechanically- or magnetically-based. Mechanical-based remote navigation systems mechanically orient the distal end of a medical device inside the body using a mechanical system, such as a pull wires or gears, after which the distal end can be advanced by pushing the proximal end. Magnetic-based remote navigation systems magnetically orient the distal end of a medical device inside the body using one or more source magnets that project a magnetic field of changeable direction inside the subject, to orient one or more magnetically responsive elements on the medical device.

### SUMMARY OF THE INVENTION

Generally, the methods of the preferred embodiments of this invention facilitate the location of the source of errant signals causing cardiac arrhythmias. In accordance with a first preferred embodiment, the leads of a standard 12 lead ECG system are placed on the subject. The positions of the leads are identified in a reference frame. A pacing catheter is placed at a known location (the location is determined for instance through Fluoro localization of the catheter tip, or through the use of a location sensor placed at the catheter tip and connected to a localization system, etc.) and is used to induce an arrhythmia. The result is the identification of a source area on the surface of the heart by means of the ECG signals recorded by the 12 leads. The pacing catheter can then be traversed over the source area, preferably using a remote navigation system, and pacing at each of a plurality of locations in the source area. The ECG signals resulting from pacing at each location in source area is compared with the ECG signal from a spontaneous arrhythmia. The location from which pacing results in an ECG the same as or closest to the ECG from a spontaneous arrhythmia is the source of the arrhythmia.

Once the source is identified, then the arrhythmia can be treated by either destroying the source, or by isolating the source. This can be conveniently done with the remote navigation system, which merely needs to return an ablation catheter (which can be the same as the pacing catheter) to the identified location. The tissue is ablated, and the arrhythmia eliminated. Alternatively, a line of ablation is formed around the source, blocking the conduction of the errant signals from the source.

### BRIEF DESCRIPTION OF THE DRAWINGS

FIG. 1 is a flow chart illustrating a preferred embodiment of the methods of this invention;

FIG. 2 is a flow chart illustrating another preferred embodiment of the methods of this invention;

FIG. 3 is a diagram showing the typical placement of the precordial leads of a 12 lead ECG;

FIG. 4 is a diagram of a heart illustrating the calibration of the 12 lead ECG for localizing an arrhythmia;

FIG. 5 is an example of an ECG showing a provoked arrhythmia which can be used to acquire the 12 lead vectors to localize the source of the arrhythmia;

FIG. 6 is diagram of the heart showing an example the source location identified from the ECG signals, with a plurality of possible pacing points therein;

FIG. 7 is a diagram of the heart showing resulting ECG signals from pacing from selected points within the identified source location;

FIG. 8 is a schematic diagram illustrating the comparison between an ECG signal from pacing with an ECG signal of a spontaneous arrhythmia.

### DETAILED DESCRIPTION OF PREFERRED EMBODIMENTS

Embodiments of the methods of this invention provide for the mapping of arrhythmias, and in particular the localization of the source of arrhythmias. Once the source is located, the arrhythmia can be treated, either with direct ablation in which an ablation device is returned to the mapped location that is identified as the source of the arrhythmias, or by isolation, where conduction paths from the source are blocked by lines of ablation.

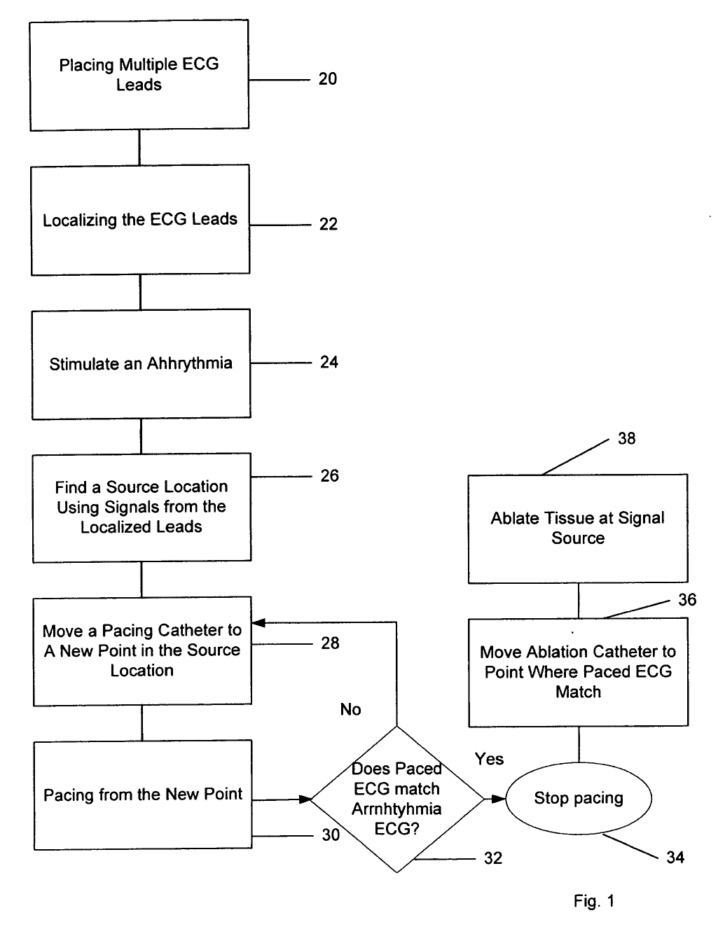

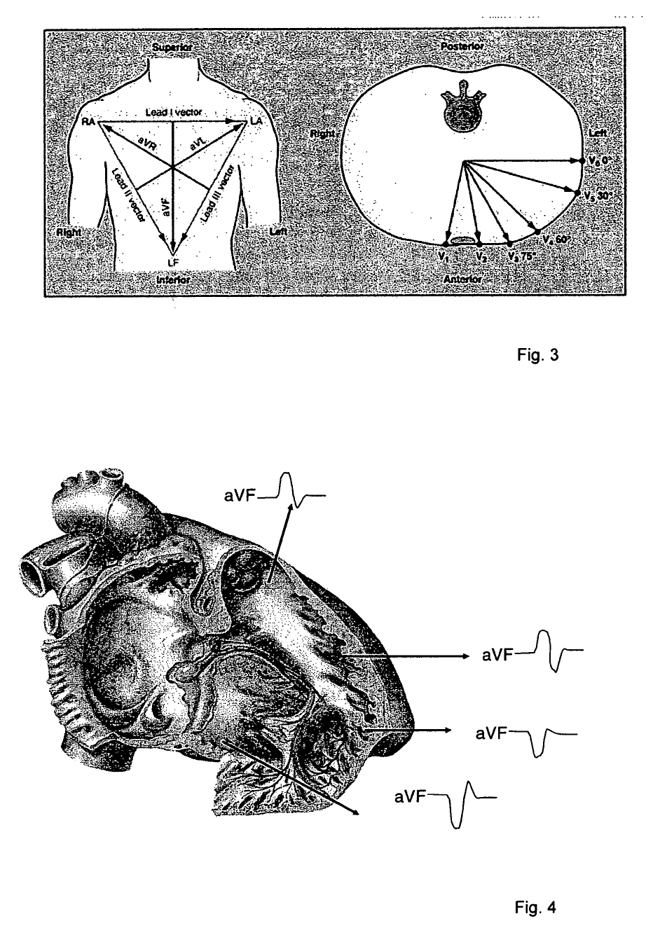

One preferred embodiment is shown in FIG. 1. As shown in FIG. 1, at step 20 the leads for a conventional 12 lead ECG are placed on the subject. A typical arrangement for the precordial leads is shown in FIG. 3. At step 22 the ECG leads are localized in a single reference frame, so that the relative positions of the leads are known. This can be done by including a localization element in each lead which can be localized, for example with an RF localization system. Alternatively, a localizing wand having a localizing element can be temporarily touched to each lead to localize the lead, for example with an RF localization system. The leads were preferably localized in the same frame or reference as a remote navigation system, or in a frame of reference with known relationship to the remote navigation system. Thus the localization system may be provided in conjunction with a remote navigation system, in which case the leads are conveniently localized in a common reference frame with the localization system.

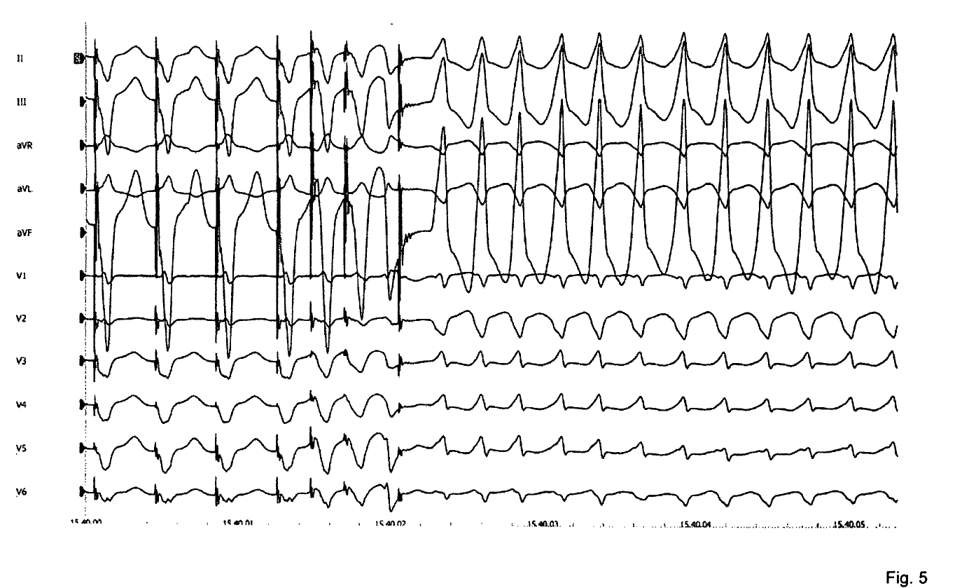

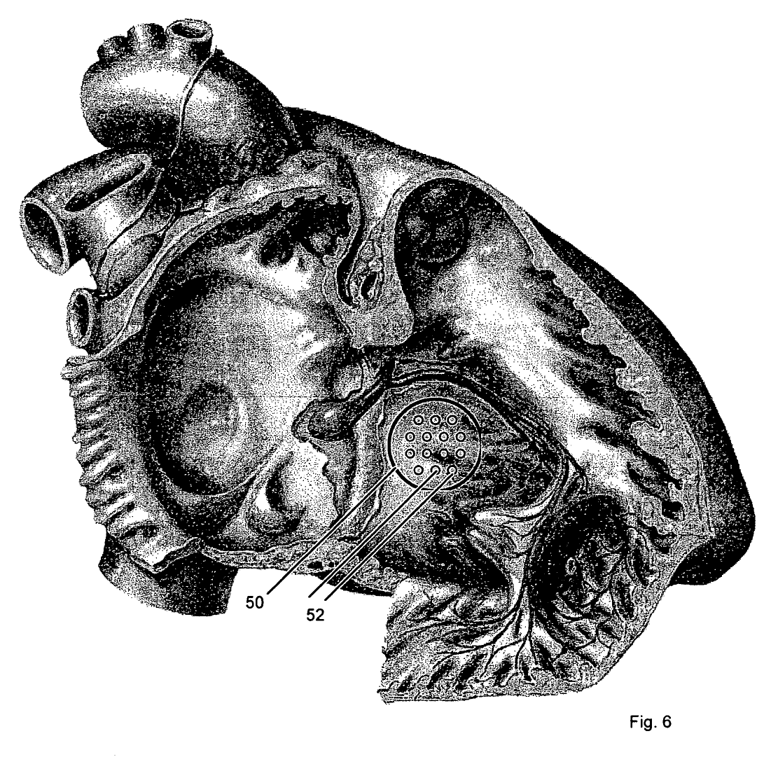

After the leads are localized, the leads can be calibrated by pacing the heart at one or more landmark locations, as shown schematically in FIG. 4, and then trying to localize the signal from the known landmark location with the pacing leads. The result is a way to localize a signal source to a small area or region using the packing leads from the 12 lead ECG system.

At step 24 an arrhythmia is stimulated with a pacing catheter. Alternatively, the subject could be monitored until an arrhythmia occurs naturally. The ECG signal corresponding to the arrhythmia is stored for later comparison, as described below. At step 26 the source location of the arrhythmia is located using the signals from the localized leads. The result, as illustrated in FIG. 5 is a generally circular area 50 on the surface of the heart that probably includes the origin of the arrhythmia.

The leads were preferably localized in the same frame or reference as a remote navigation system, or in a frame of reference with known relationship to the remote navigation system. The remote navigation system is preferably a mechanical or magnetic navigation system, although the methods could be implemented with any remote navigation system capable of remotely orienting the distal end of medical device, in response to the input of one or more control variables. Mechanical navigation systems typically employ a sleeve or collar for orienting the end of a medical device that telescopes there through. Mechanical elements such as push wires, pull wires, or other devices orient the sleeve or collar. One example of such a device is disclosed in U.S. patent application Ser. No. 10/378,547, filed Mar. 3, 2003, entitled Guide for Medical devices, which is a continuation of Ser. No. 09/875,279, filed Jun. 6, 2001, now U.S. Pat. No. 6,529,761, the disclosures of which are incorporated herein by reference. Magnetic navigation systems typically employ one or more external source magnets for creating a magnetic field in a selected direction which acts upon one or more magnetically responsive elements incorporated into the medical device to orient the distal end of the medical device. Such systems are presently available from Stereotaxis, Inc., St. Louis, Mo. Of course a remote navigation is not required to implement the present invention, and the pacing catheter could be manually moved, and localized with a localization system.

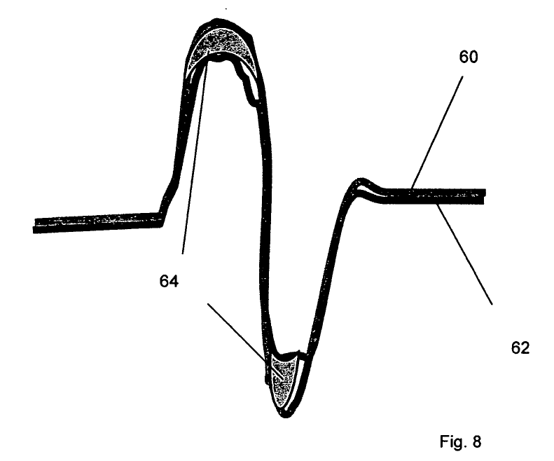

A pacing device is navigated to the probable location 50 of the source of the arrhythmia, and at step 28 the pacing device is navigated to a particular point 52 in the area 50. At step 30 the heart is paced from a particular point 52. At 32 the ECG from the pacing is compared with the ECG recorded during an arrhythmia. If the two ECGs are substantially the same, then the point is most probably the origin of the arrhythmia. If the two ECG are not similar then at step 28 the pacing device is moved to another point. Steps 28, 20 and 32 are repeated until the closest match is found and than at 34 pacing stops. FIG. 8 illustrates a comparison between the ECG signal 62 resulting from pacing at one of the points 52, and the ECG signal 60 during arrhythmia. The smaller the area 64 between these two signals, to closer the corresponding pacing point 52 is to the origin of the arrhythmia. The location of a particular pacing point 52 is preferably already known to the remote navigation system or it could be determined using a localization system, and preferably the same localization system used to localize the ECG leads.

Thereafter, as shown in FIG. 1, at 36 an ablation device (which can be the same as the pacing device) can be moved to the point corresponding to the arrhythmia source, and at 38 the tissue at the arrhythmia source is ablated.

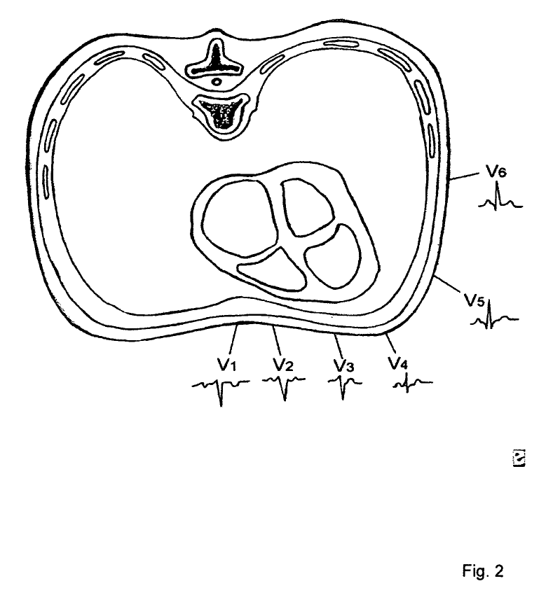

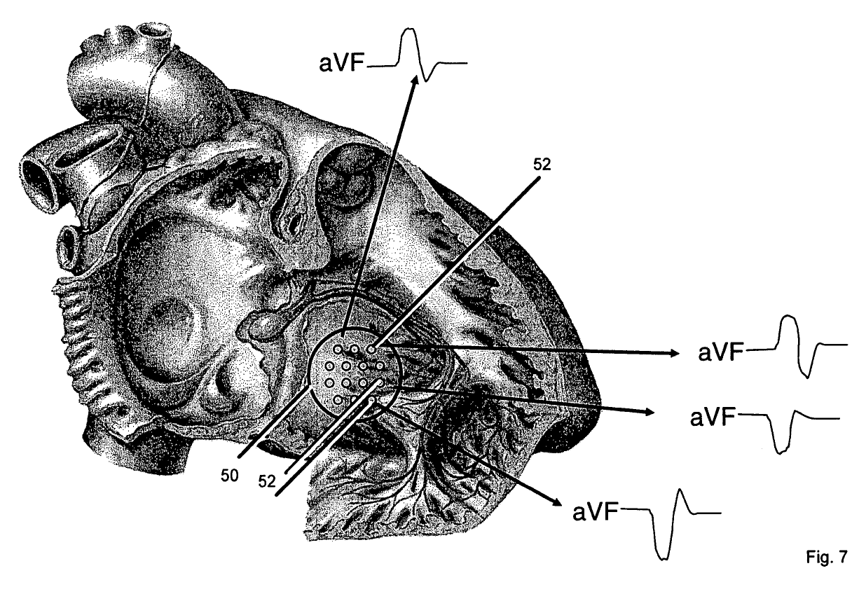

Alternatively, as shown in FIG. 2 the steps 28 of moving the pacing catheter, 30 of pacing from the point; and 32 of measuring the ECG are continued until the entire source location has been pace-mapped. FIG. 7 illustrates the differing ECG signals resulting from pacing at the various locations 52 in the area 50. Then at 40 the best fit between the pace-mapped ECGs and the arrhythmia ECG is found, and the corresponding point identified as the arrhythmia site. One way of comparing these ECG signals is shown in FIG. 8, and described above, but any method for comparing these signals and identifying the closest signal form to the arrhythmia ECG can be used. The location of the particular point 52 in the area corresponding to the origin of the arrhythmia is already known to the remote navigation system, but could also be determined with any medical localization system,

Thereafter, as shown in FIG. 2, at 36 an ablation device (which can be the same as the pacing device) can be moved to the point corresponding to the arrhythmia source, and at 38 the tissue at the arrhythmia source is ablated.

Operation

In operation, the ECG units are placed on the subject, and those leads are localized in a common reference frame (preferably the reference frame of a remote navigation system). An arrhythmia is triggered with a pacing catheter, and the signals from the plurality of leads and their known relative locations are used to determine a general location of the source of the arrhythmia.

Alternatively the pacing catheter tip is localized using standard methods. The pacing device is successively navigated to a grid of points covering the location of the source of the arrhythmia, preferably using the remote navigation system. At each point a pacing signal is applied, and the paced ECG is recorded. After the source location has been pace-mapped in this manner, the resulting pace maps are compared with an ECG during an arrhythmia. The point corresponding to closest matching pace-map is identified as the source of the arrhythmia.

Once the source is identified, it can be ablated or isolated to treat the arrhythmia. The remote navigation system can be used to return the pacing device to the identified point. The pacing catheter can then be used to ablate tissue to treat arrhythmia. The remote navigation system can be used to facilitate this navigation. Alternatively, a separate ablation catheter can be introduced and used to perform the ablation.

What is claimed is:

1. A method of mapping ventricular arrhythmias in a subject, the method comprising:
placing a plurality of ECG leads on the subject;
triggering an arrhythmia with a pacing catheter, determining the location of the pacing catheter and recording the ECG signals from the plurality of leads;
successively navigating the electrode on a catheter to each of a plurality of points in the general location of the source of the arrhythmia site, applying electrical stimulation to each site via the electrode, and recording the resulting paced ECG;
comparing each paced ECG from each point with an ECG during the arrhythmia to identify the point where the paced ECG most closely corresponds to the ECG during arrhythmia.

2. The method according to claim 1 wherein the step of using the plurality of leads to determine a general location of the arrhythmia comprises locating each of the leads in a frame of reference; identifying the general location of the site of the source of the arrhythmia by processing the signals from each lead and their relative locations.

3. The method according to claim 2 wherein the step of using the plurality of leads to determine the general location of the arrhythmia comprises calibrating the ECG leads by localizing pacing signals from known locations.

4. The method according to claim 1 wherein the step of successively navigating the electrode on a catheter to each a plurality of points is done with a remote navigation system.

5. The method according to claim 4 wherein remote navigation system is a mechanical system that remotely orients the distal end of medical device.

6. The method according to claim 4 wherein the remote navigation system is a magnetic system that remotely orients the distal end of a medical device having a magnetically responsive element therein.

7. The method according to claim 6 wherein the stop of using the plurality of leads to determine a general location of the arrhythmia comprises locating each of the leads in the frame of reference of the remote navigation system; identifying the general location of the site of the source of the arrhythmia by processing the signals from each lead and their relative locations.

8. The method according to claim 1 further comprising ablating tissue at the point to ablate tissue at the source of the arrhythmia.
